(19) United States
(12) Patent Application Publication
(10) Pub. No.: US 2023/0083143 A1
(43) Pub. Date: Mar. 16, 2023

(54) SYSTEM FOR PROVIDING HEALTH INFORMATION THROUGH CUSTOMIZED SEARCH THAT REFLECTS INDIVIDUAL HEALTH CONDITIONS, AND OPERATION METHOD THEREOF

(71) Applicant: NUTRITIONCOURT, Seoul (KR)

(72) Inventor: Yea Hang LEE, Seoul (KR)

(21) Appl. No.: 17/939,947

(22) Filed: Sep. 7, 2022

(30) Foreign Application Priority Data
Sep. 8, 2021 (KR) 10-2021-0119460

(51) Int. Cl. G16H 20/60; G06F 16/903

(52) U.S. Cl. CPC G16H 20/60; G06F 16/90335

(57) ABSTRACT

A system for providing health information through a customized search that reflects individual health conditions includes the steps of: acquiring health condition information of a user, wherein the health condition information includes an analysis result of at least one test item; selecting at least one among recommended nutrients and restricted nutrients corresponding to at least one of the test items according to the analysis result; receiving a customized search request from the user, wherein the customized search request includes a search word corresponding to at least one search target among a food, a food material, a nutrient, and a health functional food; and providing health information including information on the search target and whether the user and the search target match based on at least one among the recommended nutrients and the restricted nutrients and the search word in response to the customized search request.

### CROSS-REFERENCE TO RELATED APPLICATION(S)

The present application claim priority to Korean patent application No. 10-2021-0119460 filed on Sep. 8, 2021, the entire disclosure of which is hereby incorporated by reference.

### BACKGROUND OF THE INVENTION

### Field of the Invention

The present invention relates to a system for providing health information through a customized search that reflects individual health conditions, and an operation method thereof.

### Background of the Related Art

Interest in various nutrients that humans should take for life has become a particularly important issue to modern people, and in recent years, interest in nutrients necessary for human bodies is increasing in accordance with the trend that values quality of life.

In modern times, information on the nutrients is generally provided through Internet portal sites. However, this method is nothing but a process of searching for and providing general information common to a plurality of users, such as information on the nutrients contained in a food material, a food, a health functional food, or the like, information on the effects of the nutrients (disease improvement, prevention effect, and the like), information on calorie, and the like, when a user simply inputs a name of a food material, a food, a health functional food, or the like in an Internet portal site.

Although the nutrients required for each user are different due to the user's individual health conditions, the risk of a specific disease in the future based thereon, or the like, there is a limitation in that such universal and general nutritional information does not reflect the characteristics of individual users at all.

Therefore, the need of a technique for providing health information that reflects the characteristics of individual users through a customized search is gradually increasing so that these conventional limitations and problems can be solved.

### SUMMARY OF THE INVENTION

Therefore, the present invention has been made in view of the above problems, and it is an object of the present invention to provide a health information providing system and an operation method thereof, which can provide a user with health information through a customized search that reflects individual health conditions.

To accomplish the above object, according to one aspect of the present invention, there is provided an operation method of a system for providing health information through a customized search that reflects individual health conditions, the method comprising the steps of: acquiring health condition information of a user, wherein the health condition information includes an analysis result of at least one test item; selecting at least one among recommended nutrients and restricted nutrients corresponding to at least one of the test items according to the analysis result; receiving a customized search request from the user, wherein the customized search request includes a search word corresponding to at least one search target among a food, a food material, a nutrient, and a health functional food; and providing health information including information on the search target and whether the user and the search target match based on at least one among the recommended nutrients and the restricted nutrients and the search word in response to the customized search request.

According to an embodiment of the present invention, there is provided a system for providing health information through a customized search that reflects individual health conditions, the system comprising: at least one processor; and a memory for storing a program that can be executed by the processor, wherein the processor, by executing the program, acquires health condition information of a user including an analysis result of at least one test item, selects at least one among recommended nutrients and restricted nutrients corresponding to at least one of the test items according to the analysis result, receives, from the user, a customized search request including a search word corresponding to at least one search target among a food, a food material, a nutrient, and a health functional food, and provides health information including information on the search target and whether the user and the search target match based on at least one among the recommended nutrients and the restricted nutrients and the search word in response to the customized search request.

### BRIEF DESCRIPTION OF THE DRAWINGS

In order to further fully understand the drawings cited in the Detailed Description of the Invention, a brief description of each drawing is provided.

FIG. 1 is a view for explaining a health information providing system according to an embodiment of the present invention.

FIG. 2 is a sequence diagram illustrating an operation process of a health information providing system according to an embodiment of the present invention.

FIG. 3 is a flowchart illustrating an operation method of a health information providing server according to an embodiment of the present invention.

FIG. 4 is a view for explaining step S320 in FIG. 3.

FIG. 5 is a flowchart illustrating an embodiment of step S350 in FIG. 3.

FIG. 6 is a flowchart illustrating an embodiment of step S350 in FIG. 3.

FIG. 7 is a view for explaining an exemplary operation of a health information providing system according to an embodiment of the present invention.

FIG. 8 is a block diagram showing the configuration of a health information providing system according to an embodiment of the present invention.

### DETAILED DESCRIPTION OF THE PREFERRED EMBODIMENT

Hereinafter, preferred embodiments of the present invention will be described in detail with reference to the accompanying drawings. At this point, it should be noted that the same components in the accompanying drawings are denoted by the same reference numerals as much as possible. In addition, detailed descriptions of well-known functions and configurations that may obscure the gist of the present invention will be omitted.

Some embodiments of the present invention may be represented by functional block components and various processing steps. Some or all of the functional blocks may be implemented in various numbers of hardware and/or software components that perform specific functions. For example, the functional blocks of the present invention may be implemented by one or more microprocessors or by circuit components for a predetermined function. In addition, for example, the functional blocks of the present invention may be implemented in various programming or scripting languages. The functional blocks may be implemented as an algorithm executed in one or more processors. In addition, the present invention may employ conventional techniques for the purpose of electronic environment setting, signal processing, and/or data processing.

In addition, the terms such as “ . . . unit”, “module”, and the like described in this specification mean a unit that processes at least one function or operation, and may be implemented as hardware, software, or a combination of hardware and software. The “units” and “modules” are stored in an addressable storage medium and may be implemented by a program that can be executed by a processor.

For example, the “units” and “modules” may be implemented by components such as software components, object-oriented software components, class components, and task components, and processes, functions, attributes, procedures, subroutines, segments of program codes, drivers, firmware, micro codes, circuits, data, databases, data structures, tables, arrays, and variables.

Throughout the specification, when a part is “connected” to another part, it includes a case of being “indirectly connected” to another part with intervention of a device interposed therebetween, as well as a case of being “directly connected”. Throughout the specification, when a part “includes” a certain component, it means that other components may be further included, rather than excluding other components, unless otherwise stated.

In addition, connection lines or connection members between the components shown in the drawings merely exemplify functional connections and/or physical or circuit connections. In an actual device, a connection between components may be shown by various replaceable or added functional connections, physical connections, or circuit connections.

The terms used in the present invention are used only to describe specific embodiments, and are not intended to limit the present invention. A singular expression includes a plural expression unless the context clearly dictates otherwise. It should be understood that in the present invention, the terms such as “include”, “have”, and the like are intended to designate presence of a feature, a number, a step, an operation, a component, a part, or a combination thereof described in the specification, and do not preclude the possibility of the presence or addition of one or more other features, numbers, steps, operations, components, parts, or combinations thereof. That is, the description of “including” a specific configuration in the present invention does not exclude configurations other than the corresponding configuration, and it means that additional configurations may be included in the embodiments of the present invention or the scope of the technical spirit of the present invention.

Some components of the present invention are not essential components for performing essential functions in the present invention, but may be optional components for merely improving performance. The present invention may be implemented by including only the components essential for implementing the essence of the present invention, excluding the components used only for performance improvement, and a structure including only the essential components, excluding the optional components used for improving performance, is also included in the scope of the present invention.

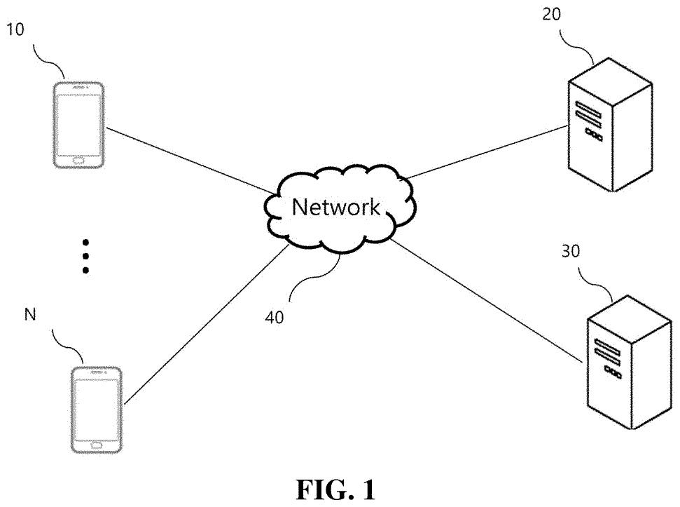

FIG. 1 is a view for explaining a health information providing system according to an embodiment.

Referring to FIG. 1, a health information providing system according to an embodiment may operate including terminals 10 to N, a health information providing server 20, a database server 30, and a network 40.

The terminals 10 to N may include all types of devices that may access the network 40. For example, the terminals 10 to N may include a smart phone, a tablet computer, a PC, a notebook computer, a home appliance device, a medical device, a camera, a wearable device, and the like. In an embodiment, the terminals 10 to N may receive customized nutritional information from the health information providing server 20.

In an embodiment, the terminals 10 to N may provide a predetermined user interface, receive personal information (identification information, age, gender, weight, allergy, medication, and the like) from a user through a user input, and transmit the personal information to the health information providing server 20 and/or the database server 30 through the network 40.

The health information providing server 20 provides computing resources, storage resources, and the like for providing customized nutritional information to a client through the network 40. Here, the client may include the terminals 10 to N. In an embodiment, the health information providing server 20 may include various types of servers, such as an application server, a control server, a data storage server, a server for providing a specific function, and the like. In addition, the health information providing server 20 may handle a process alone, or a plurality of servers may handle a process in cooperation.

In an embodiment, the health information providing server 20 may acquire health condition information of a user, select recommended nutrients and/or restricted nutrients based on the health condition, and create a customized database, and when the user inputs a search word (the name of a food, a food material, a health functional food, a nutrient, or the like) of a predetermined search target, the health information providing server 20 may provide customized health information to the terminal 10 based on the search word.

The database server 30 stores data needed for the operation of the health information providing system. In an embodiment, the database server 30 may be directly or indirectly operated and managed by at least one medical institution (hospital, examination institution, public health center, or the like) and/or national institution (e.g., National Health Insurance Service, Ministry of Food and Drug Safety, or the like) for maintenance of data, and may be configured to transfer corresponding data to a user or a business operator having a predetermined qualification. In an embodiment, the database server 30 may store health condition information (medical checkup information, medical treatment information, and the like) of a user. The information stored in the database server 30 may be transferred in response to a request of the health information providing server 20 and used in the process of providing the customized nutritional information.

The network 40 may include all types of networks that the terminals 10 to N, the health information providing server 20, and the database server 30 may access, such as the Internet, an intranet, an extranet, a local area network (LAN), a metropolitan area network (MAN), a wide area network (WAN), and the like.

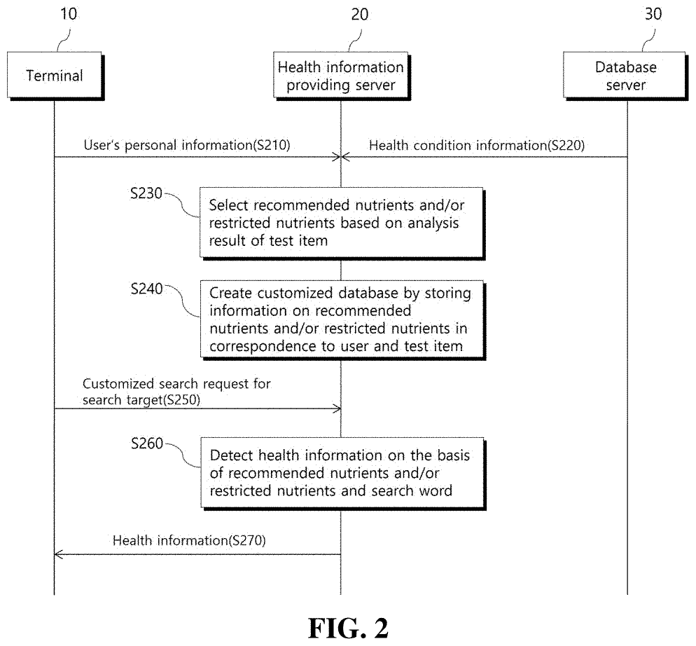

FIG. 2 is a sequence diagram illustrating an operation process of a health information providing system according to an embodiment.

Referring to FIG. 2, first, the health information providing server 20 receives personal information and/or medical questionnaire information of a user from the terminal 10 at step S210, and receives health condition information including medical checkup information and/or medical treatment information of the user at step 220. Here, the medical checkup information may include information on an analysis result (or test result) of the user for at least one test item of a test performed at a medical institution, and the medical treatment information may include diagnostic information or medication information that determines the health condition, presence of a disease, and the like of the user determined by the medical institution based on the medical checkup information.

According to embodiments, only one of steps S210 and S220 may be performed.

Thereafter, at steps S230 and S240, the health information providing server 20 may select recommended nutrients and/or restricted nutrients for each test item based on the analysis result of at least one test item included in the health condition information, and create a customized database by storing information on the recommended nutrients and/or restricted nutrients in correspondence to the user and the test items. At this point, corresponding information may be stored in the database server 30.

At step S250, the health information providing server 20 may receive a customized search request for a search target from the terminal 10. At this point, the search target may be a food material, a nutrient, a food, and/or a health functional food, and the customized search request may include a search word corresponding to the search target.

Thereafter, at step S260, the health information providing server 20 may detect health information customized to the user on the basis of the recommended nutrients and/or the restricted nutrients and the search word.

When the search target is a food, a food material, or a health functional food, the health information providing server 20 may determine whether the search target is appropriate for the user by comparing the nutrients contained in the search target with the recommended nutrients and/or restricted nutrients.

Meanwhile, when the search target is a nutrient, the health information providing server 20 may determine whether the search target is appropriate according to whether the nutrient corresponds to the recommended nutrients or the restricted nutrients. In addition, the health information providing server 20 may detect at least one product including the nutrient of the search target as a nutrient contained the product, and determine whether each of the products is appropriate for the user by comparing the nutrients contained in each product with the recommended nutrients and/or the restricted nutrients.

Thereafter, at step S270, the health information may be provided to the terminal 10 and displayed to the user. At this point, the health information may include basic information of the search target, nutrients contained therein, information on whether it is appropriate for the user, and the like.

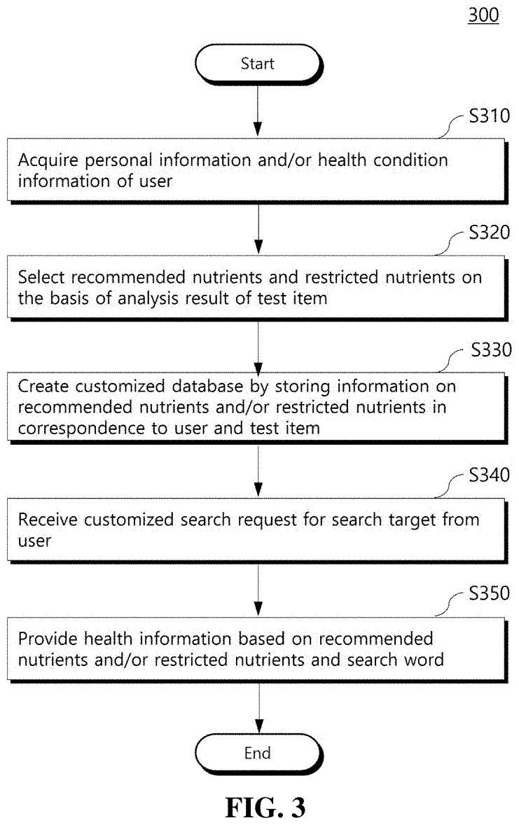

FIG. 3 is a flowchart illustrating an operation method of a health information providing server according to an embodiment of the present invention.

At step S310, the health information providing server 20 may receive personal information and/or health condition information of a user.

In an embodiment, the health information providing server 20 may receive personal information of a user from the terminal 10.

Here, the personal information may include information on at least one among the age, gender, weight, allergy, and medication of the user, as well as identification information (phone number, ID, name, date of birth, and the like) for identifying the user.

In order to perform step S310, the terminal 10 may be provided with a predetermined user interface for inputting personal information of the user. For example, the user may transfer the input personal information to the health information providing server 20 by activating a corresponding user interface by driving a dedicated application installed in the terminal 10.

In an embodiment, the health information providing server 20 may acquire health condition information of the user from the database server 30 and/or the terminal 10.

Here, the health condition information is at least one piece of information related to an index indicating a degree of health of the user, and the health condition information may include, for example, at least one among medical checkup information, medical treatment information, and lifelog information. The medical checkup information may include information on an analysis result (or analysis value) of at least one test item of a test performed at a medical institution, or the like, and the medical treatment information may include diagnostic information or medication information that determines the health condition, presence of a disease, or the like of the user based on the medical checkup information or the like. In addition, the lifelog information may include sleep-related information, eating habit information, life habit information, exercise habit information, living environment information, and the like of the user.

The health information providing server 20 may acquire health condition information of the user from the database server 30 on the basis of the user's identification information. In an embodiment, the database server 30 may be directly or indirectly operated and managed by at least one medical institution (hospital, examination institution, public health center, or the like) and/or national institution (e.g., National Health Insurance Service, or the like) for maintenance of data.

In an embodiment, the medical checkup information may include information on an analysis result of at least one blood test item. Although the blood test item may include, for example, a blood sugar level, a cholesterol level, a hemoglobin level, a liver level, a kidney function level, and the like, it is not limited thereto. In addition, according to embodiments, the medical checkup information may further include information on analysis results of various test items, such as at least one among a urine test item, a hair test item, and the like.

In an embodiment, the health information providing server 20 may acquire health condition information through a medical questionnaire of a user. That is, the health condition information may be a result determined based on a user's response to the medical questionnaire provided on the terminal 10. For example, the terminal 10 may display a medical questionnaire related to the user's health (presence of a disease, and the like) through a user interface, perform the medical questionnaire in a method of receiving a response to the medical questionnaire from the user, and transmit the result to the health information providing server 20.

At step S320, the health information providing server 20 may select at least one among recommended nutrients and restricted nutrients on the basis of the health condition information.

In an embodiment, the health information providing server 20 may select recommended nutrients and/or restricted nutrients based on the analysis result of at least one test item included in the health condition information. Here, the recommended nutrient may refer to a nutrient beneficial for improving health factors related to the test items, and the restricted nutrient may refer to a nutrient to be avoided not to worsen corresponding health factors.

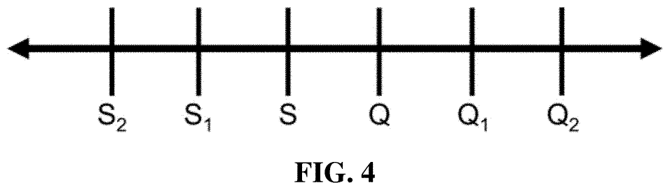

Referring to FIG. 4, the analysis result of the user for each test item may be displayed in a numerical form (i.e., analysis value), and the health information providing server 20 may select recommended nutrients and/or restricted nutrients according to the range to which the analysis value k belongs.

For example, it may be configured not to select recommended nutrients and restricted nutrients when the analysis value k for a certain test item falls within a normal range (S<k<Q), and to select recommended nutrients and/or restricted nutrients related to the health item only when the analysis value k is out of the normal range (k<S, k>Q).

In an embodiment, the health information providing server 20 may assign a different risk index according to the range to which the analysis value k belongs, and select at least one recommended nutrient and/or restricted nutrient on the basis of the risk index. For example, as shown below in Table 1, the health information providing server 20 may be configured to assign a risk index. Here, the risk index may increase in order of A to C.

TABLE 1

| Analysis value (k) | Risk index |
|---|---|
| S < k < Q (Normal) | None |
| S1 < k < S, Q < k < Q1 (Caution) | A |
| S2 < k < S1, Q1 < k < Q2 (Dangerous) | B |
| k < S2, k > Q2 (Very dangerous) | C |

According to embodiments, the health information providing server 20 may preferentially select recommended nutrients and/or restricted nutrients in relation to a health item of a high risk index, or select recommended nutrients and/or restricted nutrients only for a health item having a risk index greater than or equal to a reference value. In addition, according to embodiments, the health information providing server 20 may be configured to select a different number of recommended nutrients and/or restricted nutrients according to the risk index.

In addition, according to embodiments, when a specific nutrient corresponds to a recommended nutrient for a first test item and the same nutrient corresponds to a restricted nutrient for a second test item, the health information providing server 20 may compare the risk index of the first test item with that of the second test item, and select a corresponding nutrient as a recommended nutrient or a restricted nutrient based on a test item having a higher risk index. At this point, when the risk indexes are the same, the nutrient may not be selected as a recommended nutrient and a restricted nutrient.

In an embodiment, the health information providing server 20 may calculate a cross risk index between test items, and select recommended nutrients and/or restricted nutrients based on the cross risk index. For example, the health information providing server 20 may calculate a risk index of “diabetes due to impaired liver function” by cross-analyzing a first blood test value for blood sugar and a second blood test value related to liver function, and select recommended nutrients and/or restricted nutrients on the basis of the risk index.

At step S330, the health information providing server 20 may create a database customized to a user by storing information on the selected recommended nutrients and/or restricted nutrients in correspondence to the user and the test items (or the analysis result of the test items).

Although the health information providing server 20 may create the database by storing the information in an external database server or a cloud server, it is not limited thereto.

At step S340, the health information providing server 20 may receive a user's customized search request for a search target from the terminal 10. Here, the search target includes at least one among a food, a food material, a nutrient, and a health functional food, and the customized search request may include a search word (or keyword) corresponding to such a search target (or referring to the search target).

For example, the user may transmit a customized search request to the health information providing server 20 by inputting a search word corresponding to a search target through the user interface provided on the terminal 10.

At step S350, the health information providing server 20 may provide health information including information on a search target and whether the user and the search target match, on the basis of at least one among the recommended nutrients and the restricted nutrients and the search word.

For example, when the search target is a food, a food material, and/or a health functional food, the health information providing server 20 may determine whether it is appropriate for the user to take the search target by comparing the nutrients contained in the search target with the nutrients recommended and/or restricted for the user, and provide information thereon through the terminal 10, together with information on the nutrients contained in the search target.

In addition, for example, when the search target is a nutrient, the health information providing server 20 may detect at least one product (food, food material, and/or health functional food) containing the search target as a nutrient, determine a product appropriate for the user to take among corresponding products by comparing the nutrients contained in the product with recommended nutrients and/or restricted nutrients, and provide information thereon through the terminal 10, together with information on the nutrients contained in the product.

In an embodiment, the health information providing server 20 may further determine whether the search target is appropriate, based on the personal information of the user. For example, the health information providing server 20 may determine that the search target is inappropriate for the user when an ingredient that causes an allergy is contained in the food material, food, or health functional food, which is the search target, according to information on the allergy of the user. In addition, for example, the health information providing server 20 may determine that the search target is inappropriate for the user according to information on the medicine that user takes when an ingredient that reduces the effect of the medicine is contained in the food material, food, or health functional food, which is a search target, or when the nutrients contained in the medicine taken by the user are overlapped with the nutrients contained in the health functional food, which is a search target, as much as a predetermined proportion or more.

In an embodiment, the operation method 300 may further include the step of providing, by the health information providing server 20, purchase information of a search target or a product containing nutrients of the search target. Accordingly, the user may confirm various brands and/or sellers related to the foods, food materials, and health functional foods corresponding to the search target through the terminal 10, and make a purchase for them. At this point, the purchase information may be provided only when the search target or the product is appropriate for the user more than a predetermined level.

In an embodiment, the operation method 300 may further include the step of collecting information on the consumption pattern of the user, by the health information providing server 20, when a food, a food material, or a health functional food is ordered on the basis of the purchase information provided to the terminal 10. For example, the consumption pattern information may include data such as a user's actual purchase ratio, a list of actually purchased food materials or health functional foods, a time of intensive use by the user, and the like. In this case, the health information providing server 20 may transmit the collected consumption pattern information to other customer companies (such as online shopping malls or the like) based on the consent of the user, or analyze the user's consumption preference or the like by utilizing the consumption pattern information by himself or herself and provide customized advertisements or the like to the user.

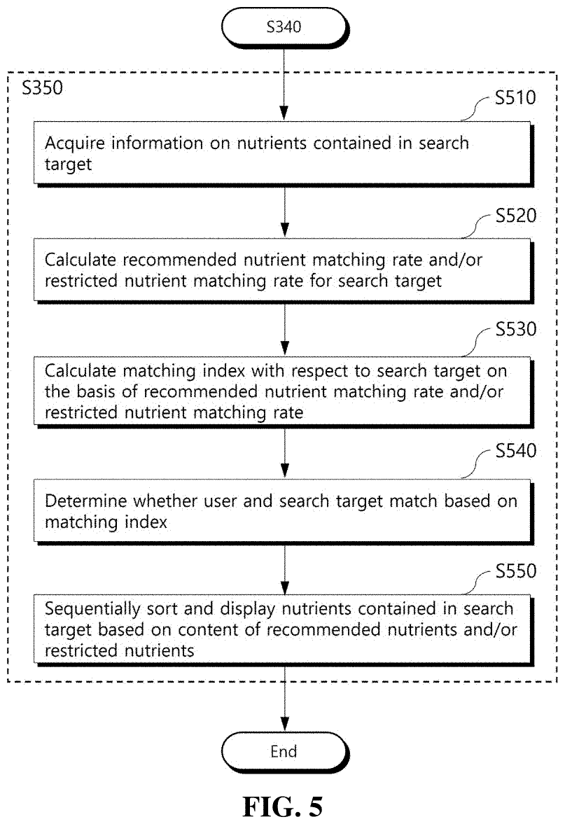

FIG. 5 is a flowchart illustrating an embodiment of step S350 in FIG. 3.

Referring to FIG. 5, the health information providing server 20 may compare the nutrients contained in the search target with recommended nutrients and/or restricted nutrients, and provide health information based on this. At this point, the search target may be at least one among a food, a food material, and a health functional food.

At step S510, when a user inputs a search word corresponding to a search target through the terminal 10, the health information providing server 20 may acquire information on the nutrients contained in the search target in correspondence thereto. The information on the contained nutrients may be acquired as the health information providing server 20 searches for information on the ingredient table of the search target from the ingredient DB or the like constructed in the database server 30.

At step S520, the health information providing server 20 may calculate a recommended nutrient matching rate and/or a restricted nutrient matching rate for the search target.

For example, the health information providing server 20 may calculate the recommended nutrient matching rate and the restricted nutrient matching rate through the following equation, respectively.

Recommended nutrient matching rate=L/N×100(%)

Restricted nutrient matching rate=M/N×100(%)

Here, L may denote the number of nutrients corresponding to the recommended nutrients among the nutrients contained in the search target, M may denote the number of nutrients corresponding to the restricted nutrients among the nutrients contained in the search target, and N may denote the total number of nutrients contained in the search target.

At step S530, the health information providing server 20 may calculate a matching index with respect to the search target on the basis of the recommended nutrient matching rate and/or the restricted nutrient matching rate.

For example, the matching index may be calculated by subtracting the restricted nutrient matching rate from the recommended nutrient matching rate. At this point, the higher the matching index, the higher the ratio of the recommended nutrients to the restricted nutrients contained in the search target. However, this method is exemplary and not limited thereto, and when the restricted nutrients matching rate is lower than or equal to a predetermined reference value, various methods such as a method of reflecting only the recommended nutrient matching rate in the matching index may be applied.

At step S540, the health information providing server 20 may determine whether the user and the search target match based on the matching index. For example, the health information providing server 20 may determine that it is appropriate for the user to take the search target when the matching index is greater than or equal to a predetermined reference value. In addition, for example, the health information providing server 20 may grant a different custom grade according to a range of numerical values to which the matching index belongs, and determine whether the user and the search target match as one of a plurality of custom grades based on this.

At step S550, the health information providing server 20 may provide the terminal 10 with health information including information on the search target and whether the user and the search target match. For example, information on the search target may include information on the nutrients contained in the search target, and information on whether the search target and the user match may include information on whether or not to recommend the user to take the search target and information on the matching index.

In an embodiment, the health information providing server 20 may sequentially sort the nutrients contained in the search target based on the content of the recommended nutrients and/or the restricted nutrients, and display the nutrients on the terminal 10.

In addition, in an embodiment, the health information providing server 20 may compare the content of the nutrients contained in the search target with the amount of the recommended nutrients and/or the restricted nutrients recommended to take daily, and preferentially sort and display nutrients of a content higher than the recommended amount.

In addition, in an embodiment, the health information providing server 20 may sequentially sort and display the nutrients contained in the search target based on the risk index of test items corresponding to the recommended nutrients and/or the restricted nutrients (i.e., sort in order of nutrients having a high risk index).

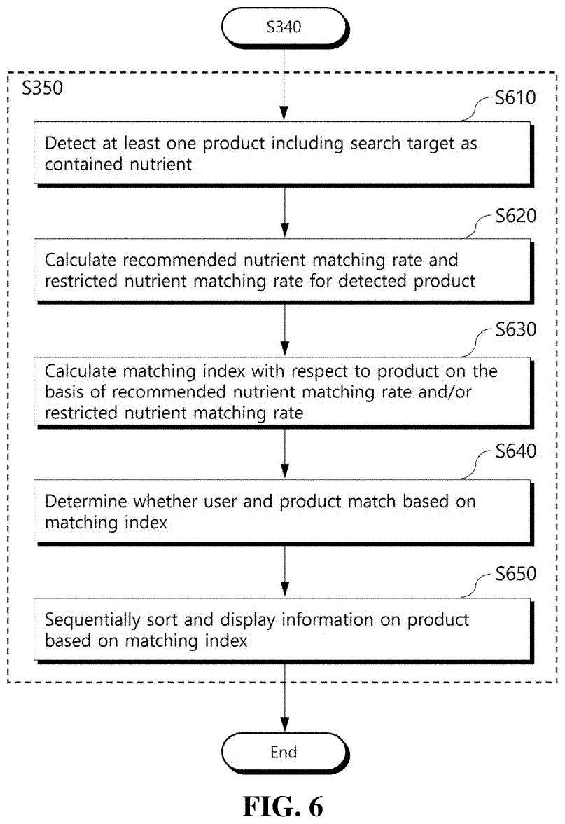

FIG. 6 is a flowchart illustrating an embodiment of step S350 in FIG. 3.

Referring to FIG. 6, the health information providing server 20 may detect at least one product containing a search target as a contained nutrient, compare the nutrients contained in the product with the recommended nutrients and/or the restricted nutrients, and provide health information based on the result of the comparison. At this point, the search target may be a nutrient, and the product may be at least one among a food, a food material, and a health functional food.

At step S610, when the user inputs a search word corresponding to the search target through the terminal 10, the health information providing server 20 may detect at least one product including the search target as a contained nutrient in response thereto. Such a product may be acquired as the health information providing server 20 searches for a product including a search target as an ingredient from the ingredient DB or the like constructed in the database server 30.

At step S620, the health information providing server 20 may calculate a recommended nutrient matching rate and a restricted nutrient matching rate for each product based on the nutrients contained in each product and the recommended nutrients and the restricted nutrients of the user.

For example, the health information providing server 20 may calculate a recommended nutrient matching rate and a restricted nutrient matching rate for each product through the following equation in the same manner as described above with reference to FIG. 5.

Recommended nutrient matching rate=L/N×100(%)

Restricted nutrient matching rate=M/N×100(%)

Here, L may denote the number of nutrients corresponding to the recommended nutrients among the nutrients contained in the product, M may denote the number of nutrients corresponding to the restricted nutrients among the nutrients contained in the product, and N may denote the total number of nutrients contained in the product.

At step S630, the health information providing server 20 may calculate a matching index between each product and the user on the basis of the recommended nutrient matching rate and/or the restricted nutrient matching rate.

For example, the matching index may be calculated by subtracting the restricted nutrient matching rate from the recommended nutrient matching rate for each product. At this point, the higher the matching index, the higher the ratio of the recommended nutrients to the restricted nutrients contained in the search target. However, this method is exemplary and not limited thereto, and when the restricted nutrients matching rate is lower than or equal to a predetermined reference value, various methods such as reflecting only the recommended nutrient matching rate in the matching index may be applied.

At step S640, the health information providing server 20 may determine whether the user and the product match based on the matching index. For example, the health information providing server 20 may determine that it is appropriate for the user to take the product when the matching index is greater than or equal to a predetermined reference value. In addition, for example, the health information providing server 20 may grant a different custom grade according to a range of numerical values to which the matching index belongs, and determine whether the user and the product match as one of a plurality of custom grades based on this.

At step S650, the health information providing server 20 may sequentially sort and display information on the product on the terminal 10 on the basis of the matching index. For example, information on the product may include information on the nutrients contained in the product, the matching index, and information on whether or not to recommend based thereon.

At this point, the health information providing server 20 may sequentially sort and display the nutrients contained in each product based on the content of recommended nutrients and/or restricted nutrients.

In an embodiment, the health information providing server 20 may sequentially sort the nutrients contained in the product based on the content of the recommended nutrients and/or the restricted nutrients, and display the nutrients on the terminal 10. In addition, in an embodiment, the health information providing server 20 may sequentially sort and display the nutrients contained in the product based on the risk index of test items corresponding to the recommended nutrients and/or the restricted nutrients (i.e., sort in order of nutrients having a high risk index).

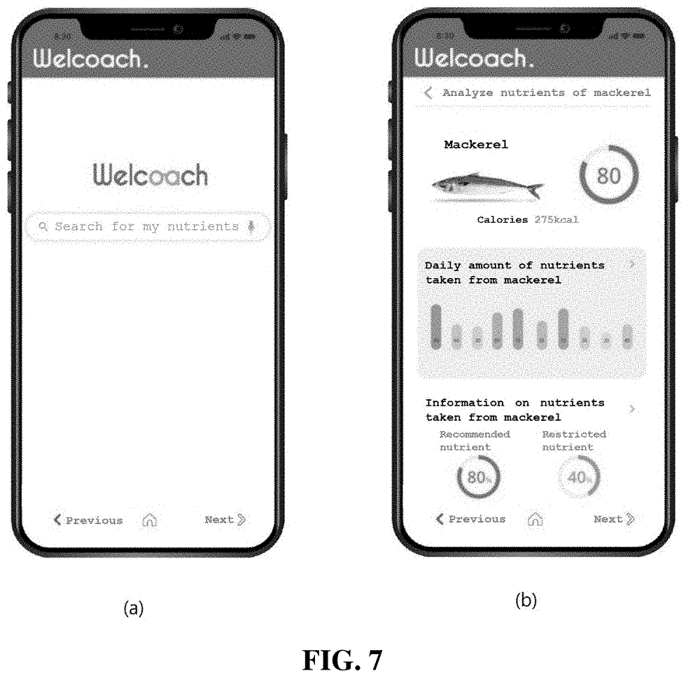

FIG. 7 is a view for explaining an exemplary operation of a health information providing system according to an embodiment of the present invention.

More specifically, FIG. 7 exemplarily shows a user interface and information provided to the terminal 10 of a user through a dedicated application according to the operation of the health information providing system. Hereinafter, through the steps S310 to S330 described above with reference to FIG. 3, it will be described in detail on the assumption that a customized database of the user is created.

First, referring to FIG. 7(a), a user may input a search word corresponding to a search target into the terminal 10 through a user interface for inputting search words. For example, the user may input the name of food material “mackerel” as a search word. When the search word is input, a customized search request may be transmitted from the terminal 10 to the health information providing server 20.

Subsequently, referring to FIG. 7(b), health information generated by the health information providing server 20 in response to the customized search request may be displayed on the terminal 10. As shown in the drawing, basic information of a food material to be searched, contained nutrients, a degree of a search target appropriate to the user (matching score), a recommended nutrient matching rate and a restricted nutrient matching rate of the search target, and the like may be provided to the terminal 10 as health information.

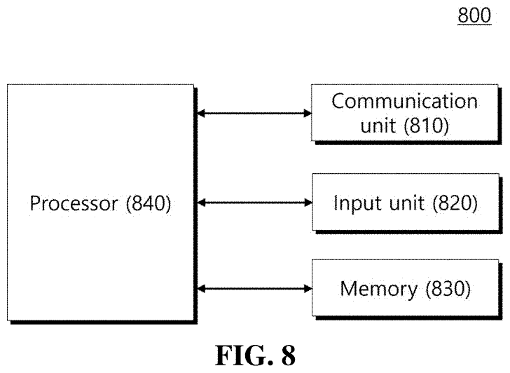

FIG. 8 is a block diagram showing the configuration of a health information providing system according to an embodiment of the present invention.

As shown in FIG. 8, the health information providing system may include a communication unit 810, an input unit 820, a memory 830, and a processor 840. However, the components of the health information providing system are not limited to the example described above. For example, the health information providing system may include more or fewer components than the components described above. In addition, at least two or more among the communication unit 810, the input unit 820, the memory 830, and the processor 840 may be implemented in the form of one chip.

The communication unit 810 may transmit and receive signals to and from an external device. The signals transmitted and received to and from the external device may include control information and data. At this point, the external device may include the terminal 10, the database server 30, and the like. The communication unit 810 may include both wired and wireless communication units. In addition, the communication unit 810 may receive signals through a wired/wireless channel and output the signals to the processor 840, and transmit the signals output from the processor 840 through a wired/wireless channel.

The input unit 820 may receive various user commands through external handling. To this end, the input unit 820 may include or connect to one or more input devices. For example, the input unit 820 may be connected to an interface for various inputs, such as a keypad, a mouse, and the like, to receive user commands. To this end, the input unit 820 may include an interface such as a Thunderbolt, as well as a USB port. In addition, the input unit 820 may receive external user commands by including various input devices such as a touch screen, buttons, and the like or in combination with the input devices.

The memory 830 may store programs and data needed for the operation of the health information providing system. In an embodiment, the memory 830 may store control information or data included in the signals transmitted and received by the health information providing system. The memory 830 may be configured as a storage medium, such as ROM, RAM, hard disk, CD-ROM, or DVD, or a combination of storage media. In addition, the memory 830 may be provided in plurality. According to an embodiment, the memory 830 may store programs for performing the operations for the health information providing system, which are embodiments of the present invention described above.

The processor 840 may control a series of processes operated by the health information providing system according to an embodiment of the present invention described above. For example, components of the health information providing system may be controlled to perform the operation of the health information providing system according to an embodiment. The processor 840 may be provided in plurality, and the processor 840 may perform the operation of the health information providing system by executing a program stored in the memory 830.

In an embodiment, the processor 840 may acquire health condition information of a user including an analysis result of at least one test item, select at least one among recommended nutrients and restricted nutrients corresponding to at least one of the test items according to the analysis result, receive, from the user, a customized search request including a search word corresponding to at least one search target among a food, a food material, a nutrient, and a health functional food, and provide health information including information on the search target and whether the user and the search target match based on at least one among the recommended nutrients and the restricted nutrients and the search word in response to the customized search request.

According to an embodiment, the processor 840 may create a customized database by storing information on at least one among the selected recommended nutrients and restricted nutrients in correspondence to the user and the test items.

According to an embodiment, the processor 840 may select at least one among the recommended nutrients and restricted nutrients based on at least one among an individual risk index of each test item calculated based on an analysis result of the test item and a cross risk index between the test items.

According to an embodiment, the processor 840 may control to receive user information including information on at least one among allergies and medications of the user, and provide health information further based on the user information.

According to an embodiment, the processor 840 may acquire information on the nutrients contained in the search target, calculate at least one among a recommended nutrient matching rate and a restricted nutrient matching rate based on the nutrients contained in the search target, the recommended nutrients, and the restricted nutrients, and determine whether the user and the search target match based on at least one among the recommended nutrient matching rate and the restricted nutrient matching rate. Here, the search target may be at least one among a food, a food material, and a health functional food.

According to an embodiment, the processor 840 may sequentially sort and display the nutrients contained in the search target based on the content of at least one among the recommended nutrients and the restricted nutrients.

According to an embodiment, the processor 840 may calculate a matching index between the user and the search target by subtracting the restricted nutrient matching rate from the recommended nutrient matching rate, and determine whether the user and the search target match based on the matching index.

According to an embodiment, the processor 840 may detect at least one product including the search target as a contained nutrient, calculate a recommended nutrient matching rate and a restricted nutrient matching rate for each product based on the nutrients contained in the product, the recommended nutrients, and the restricted nutrients, calculate a matching index between the user and each of the products by subtracting the restricted nutrient matching rate from the recommended nutrient matching rate, and determine whether the user and the product match based on the matching index. Here, the search target is a nutrient, and the product may include at least one among a food, a food material, and a health functional food.

According to an embodiment, the processor 840 may sequentially sort and display information on the plurality of products based on the matching index.

Meanwhile, the embodiment described above may be written as a program that can be executed on a computer, and may be implemented in a general-purpose digital computer that executes the program using a computer-readable medium. In addition, the structure of data used in the embodiment described above may be recorded in a computer-readable medium through various means. In addition, the embodiment described above may be implemented in the form of a recording medium including instructions that can be executed by a computer, such as a program module executed by a computer. For example, the methods implemented as a software module or algorithm are codes or program instructions that can be read and executed by a computer, and may be stored in a computer-readable recording medium.

The computer-readable medium may be any recording medium that can be accessed by a computer, and may include volatile and nonvolatile media, removable and non-removable media. Although the computer-readable medium may include magnetic storage media such as ROM, floppy disk, hard disk, and the like, and optically readable media such as CD-ROM, DVD, and the like, it not limited thereto. In addition, the computer-readable medium may include computer storage media and communication media.

In addition, a plurality of computer-readable recording media may be distributed in computer systems connected through a network, and data stored in the distributed recording media, for example, program instructions and codes, may be executed by at least one computer.

The specific implementations described in the present invention are merely embodiments and do not limit the scope of the present invention in any way. For brevity of the specification, descriptions of conventional electronic components, control systems, software, and other functional aspects of the systems may be omitted.

What is claimed is:

1. An operation method of a system for providing health information through a customized search that reflects individual health conditions, the method comprising:
acquiring health condition information of a user, wherein the health condition information includes an analysis result of at least one test item;
selecting at least one among recommended nutrients and restricted nutrients corresponding to at least one of the test items according to the analysis result;
receiving a customized search request from the user, wherein the customized search request includes a search word corresponding to at least one search target among a food, a food material, a nutrient, and a health functional food; and
providing health information including information on the search target and whether the user and the search target match based on at least one among the recommended nutrients and the restricted nutrients and the search word in response to the customized search request.

2. The method according to claim 1, further comprising creating a customized database by storing information on at least one among the selected recommended nutrients and restricted nutrients in correspondence to the user and the test item.

3. The method according to claim 1, wherein the selecting at least one among the recommended nutrients and the restricted nutrients comprises selecting at least one among the recommended nutrients and restricted nutrients based on at least one among an individual risk index of each test item, calculated based on the analysis result of the test item, and a cross risk index between the test items.

4. The method according to claim 1, further comprising receiving user information including information on at least one among allergies and medications of the user, wherein the providing the health information is performed based on the user information.

5. The method according to claim 1, wherein the search target is at least one among a food, a food material, and a health functional food, and
wherein the providing the health information comprises:
acquiring information on the nutrients contained in the search target;
calculating at least one among a recommended nutrient matching rate and a restricted nutrient matching rate based on the nutrients contained in the search target, the recommended nutrient, and the restricted nutrient; and
determining whether the user and the search target match based on at least one among the recommended nutrient matching rate and the restricted nutrient matching rate.

6. The method according to claim 5, wherein the providing health information further comprises sequentially sorting and displaying the nutrients contained in the search target based on a content of at least one among the recommended nutrients and the restricted nutrients.

7. The method according to claim 5, wherein the determining whether the user and the search target match comprises:
calculating a matching index between the user and the search target by subtracting the restricted nutrient matching rate from the recommended nutrient matching rate; and
determining whether the user and the search target match based on the matching index.

8. The method according to claim 1, wherein the search target is a nutrient, and
wherein the providing the health information comprises:
detecting at least one product including the search target as a contained nutrient, wherein the product includes at least one among a food, a food material, and a health functional food;
calculating a recommended nutrient matching rate and a restricted nutrient matching rate for each product based on the nutrients contained in the product, the recommended nutrients, and the restricted nutrients;
calculating a matching index between the user and each of the products by subtracting the restricted nutrient matching rate from the recommended nutrient matching rate; and
determining whether the user and the product match based on the matching index.

9. The method according to claim 8, wherein the providing the health information further comprises sequentially sorting and displaying information on the at least one product based on the matching index.

10. A system for providing health information through a customized search that reflects individual health conditions, the system comprising:
at least one processor; and
a memory for storing a program that can be executed by the at least one processor,
wherein the at least one processor, by executing the program, acquires health condition information of a user including an analysis result of at least one test item, selects at least one among recommended nutrients and restricted nutrients corresponding to at least one of the test items according to the analysis result, receives, from the user, a customized search request including a search word corresponding to at least one search target among a food, a food material, a nutrient, and a health functional food, and provides health information including information on the search target and whether the user and the search target match based on at least one among the recommended nutrients and the restricted nutrients and the search word in response to the customized search request.

11. A computer program stored in a non-transitory recording medium to execute the method of claim 1.
